(19) United States
(12) Patent Application Publication
(10) Pub. No.: US 2018/0221644 A1
(43) Pub. Date: Aug. 9, 2018

(54) SYSTEMS AND METHODS FOR UTILIZING DEEP BRAIN STIMULATION LOCAL EVOKED POTENTIALS FOR THE TREATMENT OF NEUROLOGICAL DISORDERS

(71) Applicant: Duke University, Durham, NC (US)

(72) Inventors: Warren M. Grill, Durham, NC (US); David T. Brocker, Durham, NC (US)

(21) Appl. No.: 15/845,064

(22) Filed: Dec. 18, 2017

Related U.S. Application Data
(63) Continuation of application No. PCT/US16/37420, filed on Jun. 14, 2016.
(60) Provisional application No. 62/182,044, filed on Jun. 19, 2015.

(51) Int. Cl. A61N 1/02; A61B 5/0484; A61N 1/05; A61N 1/36; A61B 5/0482; A61N 1/378

(52) U.S. Cl. CPC A61N 1/025; A61B 5/0484; A61N 1/0534; A61N 1/36125; A61N 1/36139; A61B 5/0482; A61N 1/378; A61N 1/36067

(57) ABSTRACT

Systems, methods, and devices are disclosed for the use of evoked potentials from the deep brain stimulation (DBS) electrode as a feedback signal to indicate lead/contact placement relative to brain targets and to enable selection or modulation of effective stimulation parameters. In some embodiments, the brain signals are recorded using specialized equipment and used to determine characteristics of the electrode position and response to stimulation. In some embodiments, the device is an implantable device.

### RELATED APPLICATIONS

This application claims priority to PCT Patent Application No. PCT/US16/37420 filed on Jun. 14, 2016, and entitled “SYSTEMS AND METHODS FOR UTILIZING DEEP BRAIN STIMULATION LOCAL EVOKED POTENTIALS FOR THE TREATMENT OF NEUROLOGICAL DISORDERS”, which claims priority to U.S. Provisional Patent Application No. 62/182,044 filed on Jun. 19, 2015 and entitled “SYSTEMS AND METHODS FOR UTILIZING DEEP BRAIN STIMULATION LOCAL EVOKED POTENTIALS FOR THE TREATMENT OF NEUROLOGICAL DISORDERS”. Each of these applications are incorporated by reference herein.

### TECHNICAL FIELD

The present disclosure relates to the technical field of using deep brain stimulation in the treatment of neurological disorders such as Parkinson's disease.

### BACKGROUND

Evoked potentials are neural signals recorded in response to a stimulus and are used to probe neural systems. For example, visual evoked potentials recorded over the occipital cortex reveal important characteristics of the visual neural circuits, and somatosensory evoked potentials assess spinal cord function, conduction times, or stimulus sensitivity. Similarly, electrically evoked potentials in the cochlear nerve and spinal cord—also called evoked compound action potentials—guide stimulation parameter selection in cochlear implants and spinal cord stimulation. Although deep brain stimulation (DBS) evoked potentials have been recorded in the cortex, recordings in the stimulated nucleus are rare due to the challenge of large stimulation artifacts saturating amplifiers and overriding the underlying neural signal.

DBS is used to treat a variety of neurological disorders and is an effective therapy for the cardinal motor symptoms of PD. The STN is the most common target for DBS in PD, and STN DBS suppresses symptoms in a frequency-dependent manner—high frequency stimulation (>100 Hz) is effective, while low frequency stimulation is not. However, the underlying mechanisms of action of DBS remain subject to debate, and it is even unclear what neural elements are responsible for mediating symptom relief.

### SUMMARY OF THE DISCLOSURE

The present disclosure provides, in part, the use of evoked potentials recorded from the deep brain stimulation (DBS) electrode as a feedback signal to indicate lead/contact placement relative to brain targets and so enable selection or modulation of stimulation parameters. In some embodiments, the brain signals are recorded using specialized equipment and used to determine characteristics of the electrode position and response to stimulation. This solves two existing problems. First, it is difficult to place a brain stimulation electrode in a small target brain area, and surgeons do not currently have real-time electrophysiological feedback. The location-specific characteristics of deep brain stimulation evoked potentials provide such a feedback signal and will improve electrode placement accuracy. Second, brain stimulation parameters, including stimulation pulse amplitude (either voltage or current), stimulation pulse duration, and stimulation pulse repetition rate (or frequency) are determined empirically by observing the effect of the stimulation on the symptoms. Symptoms can vary with time and are dependent on many factors beside the stimulation parameters. Therefore, having an electrophysiological signal that is an indicator of the stimulation's effect in the brain will be very useful for determining the proper stimulation parameters. This disclosure describes an electrophysiological signal that can be used for this purpose and describes methods for recording and using the signal in practice.

A novel amplifier configuration was developed incorporating diode clamping and amplifier blanking across three serial stages of amplification to record evoked potentials intraoperatively in the subthalamic nucleus (STN) of persons with Parkinson's disease (PD) during different frequencies of STN DBS. The responses recorded from contacts of the stimulating electrode are termed DBS local evoked potentials (DLEPs).

DLEPS were quantified in patients with STN DBS for PD, and demonstrate that DLEPs reveal the functional connectivity in the human basal ganglia, contribute directly to determining the effects of DBS in PD, and are strongly dependent on electrode location.

Disclosed herein, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a device including: a processor and memory operable to:. transfer a plurality of stimuli, receive a plurality of evoked potentials in response to the plurality of stimuli, perform analysis of the plurality of evoked potentials, determine one or more stimulation parameters based on the analysis of the plurality of evoked potentials, and communicate the one or more stimulation parameters. Other embodiments of this aspect include corresponding computer systems, apparatus, and computer programs recorded on one or more computer storage devices, each configured to perform the actions of the methods.

One general aspect includes a method including: transferring a plurality of stimuli, receiving a plurality of evoked potentials in response to the plurality of stimuli, performing analysis of the plurality of evoked potentials, determining one or more stimulation parameters based on the analysis of the plurality of evoked potentials, and communicating the one or more stimulation parameters. Other embodiments of this aspect include corresponding computer systems, apparatus, and computer programs recorded on one or more computer storage devices, each configured to perform the actions of the methods.

One general aspect includes a non-transitory computer-readable storage medium containing program instructions to cause a processor to perform a method of operating a device including: transferring a plurality of stimuli, receiving a plurality of evoked potentials in response to the plurality of stimuli, performing analysis of the plurality of evoked potentials, determining one or more stimulation parameters based on the analysis of the plurality of evoked potentials, and communicating the one or more stimulation parameters. Other embodiments of this aspect include corresponding computer systems, apparatus, and computer programs recorded on one or more computer storage devices, each configured to perform the actions of the methods.

One general aspect includes a system including: a device including: a processor and memory. The system also includes transfer a plurality of stimuli. The system also includes receive a plurality of evoked potentials in response to the plurality of stimuli. The system also includes perform analysis of the plurality of evoked potentials. The system also includes determine one or more stimulation parameters based on the analysis of the plurality of evoked potentials. The system also includes communicate the one or more stimulation parameters to a client; and the client operable to: receive the one or more stimulation parameters from the device, and present the the one or more stimulation parameters on a display of a second device executing the client. Other embodiments of this aspect include corresponding computer systems, apparatus, and computer programs recorded on one or more computer storage devices, each configured to perform the actions of the methods. In some embodiments, the device is an implantable device.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a graphical depiction of a hand held device to deliver stimulation and record DLEPs in the intraoperative environment according to some embodiments of the present disclosure;

FIG. 2 is a system for recording neural signals related to DBS lead implantation according to some embodiments of the present disclosure;

FIG. 3 is a block diagram schematic of DLEP recording capabilities included in an implantable pulse generator according to some embodiments of the present disclosure;

FIG. 4 is a tabular representation of subject information according to some embodiments of the present disclosure;

FIG. 5 is a tabular representation of a comparison between contact locations predicted from the DLEP signal and imaging analysis according to some embodiments of the present disclosure;

FIG. 6A is a graphical representation of short latency DLEPs evoked by different DBS frequencies for Subject 1 according to some embodiments of the present disclosure;

FIG. 6B is a graphical representation of short latency DLEPs evoked by different DBS frequencies for Subject 2 according to some embodiments of the present disclosure;

FIG. 6C is a graphical representation of short latency DLEPs evoked by different DBS frequencies for Subject 3 according to some embodiments of the present disclosure;

FIG. 6D is a graphical representation of short latency DLEPs evoked by different DBS frequencies for Subject 4 according to some embodiments of the present disclosure;

FIG. 6E is a graphical representation of short latency DLEPs evoked by different DBS frequencies for Subject 5 according to some embodiments of the present disclosure;

FIG. 6F is a graphical representation of short latency DLEPs evoked by different DBS frequencies for Subject 6 according to some embodiments of the present disclosure;

FIG. 6G is a graphical representation of short latency DLEPs evoked by different DBS frequencies for Subject 7 according to some embodiments of the present disclosure;

FIG. 6H is a graphical representation of short latency DLEPs evoked by different DBS frequencies for Subject 8 according to some embodiments of the present disclosure;

FIG. 6I is a graphical representation of short latency DLEPs evoked by different DBS frequencies for Subject 9 according to some embodiments of the present disclosure;

FIG. 7A is a graphical representation of long latency DLEPs evoked by different DBS frequencies for Subject 1 according to some embodiments of the present disclosure;

FIG. 7B is a graphical representation of long latency DLEPs evoked by different DBS frequencies for Subject 2 according to some embodiments of the present disclosure;

FIG. 7C is a graphical representation of long latency DLEPs evoked by different DBS frequencies for Subject 3 according to some embodiments of the present disclosure;

FIG. 7D is a graphical representation of long latency DLEPs evoked by different DBS frequencies for Subject 4 according to some embodiments of the present disclosure;

FIG. 7E is a graphical representation of long latency DLEPs evoked by different DBS frequencies for Subject 5 according to some embodiments of the present disclosure;

FIG. 7F is a graphical representation of long latency DLEPs evoked by different DBS frequencies for Subject 6 according to some embodiments of the present disclosure;

FIG. 7G is a graphical representation of long latency DLEPs evoked by different DBS frequencies for Subject 7 according to some embodiments of the present disclosure;

FIG. 7H is a graphical representation of long latency DLEPs evoked by different DBS frequencies for Subject 8 according to some embodiments of the present disclosure;

FIG. 7I is a graphical representation of long latency DLEPs evoked by different DBS frequencies for Subject 9 according to some embodiments of the present disclosure;

FIG. 8A is a graphical representation of a computational modeling revealing the neural origin of the DLEPs according to some embodiments of the present disclosure;

FIG. 8B is a graphical representation of a computational modeling including subthalamic and globus pallidus neurons and cortical axons of the hyperdirect pathway according to some embodiments of the present disclosure;

FIG. 8C is a graphical representation of a 45 Hz DBS with placement of contacts within the STN and ventral/dorsal to STN according to some embodiments of the present disclosure;

FIG. 8D is a graphical representation of a 130 Hz DBS with placement of contacts within the STN and ventral/dorsal to STN according to some embodiments of the present disclosure;

FIG. 8E is a graphical representation of post-stimulus time histograms (PSTHs) for STN firing during 45 Hz according to some embodiments of the present disclosure;

FIG. 8F is a graphical representation of post-stimulus time histograms (PSTHs) for STN firing during 130 Hz according to some embodiments of the present disclosure;

FIG. 8G is a graphical representation of GPe spiking during 45 Hz according to some embodiments of the present disclosure;

FIG. 8H is a graphical representation of GPe spiking during 130 Hz according to some embodiments of the present disclosure;

FIG. 9A is a graphical representation of l²-norm of the difference vector between individual DLEPs and the mean DLEP revealed time-dependent changes in the DLEP exclusively for 130 Hz DBS according to some embodiments of the present disclosure;

FIG. 9B is a graphical representation of the amplitude of the P1 peak with successive pulses over the first 100 ms according to some embodiments of the present disclosure;

FIG. 9C is a graphical representation of magnitudes and latencies of the P1 and N1 deflections varied across time after a second successive pulse according to some embodiments of the present disclosure;

FIG. 9D is a graphical representation of magnitudes and latencies of the P1 and N1 deflections varied across time after a third successive pulse according to some embodiments of the present disclosure;

FIG. 9E is a graphical representation of magnitudes and latencies of the P1 and N1 deflections varied across time after a fourth successive pulse according to some embodiments of the present disclosure;

FIG. 9F is a graphical representation of magnitudes and latencies of the P1 and N1 deflections varied across time after a fifth successive pulse according to some embodiments of the present disclosure;

FIG. 9G is a graphical representation of changes in P1 amplitude and latencies using 5-15 second stimulation intervals according to some embodiments of the present disclosure;

FIG. 9H is a graphical representation of changes in P1 amplitude and latencies using 50-60 second stimulation intervals according to some embodiments of the present disclosure;

FIG. 10A is a graphical representation of power spectra of local field potentials for Subject 1 according to some embodiments of the present disclosure;

FIG. 10B is a graphical representation of power spectra of local field potentials for Subject 2 according to some embodiments of the present disclosure;

FIG. 10C is a graphical representation of power spectra of local field potentials for Subject 3 according to some embodiments of the present disclosure;

FIG. 10D is a graphical representation of power spectra of local field potentials for Subject 4 according to some embodiments of the present disclosure;

FIG. 10E is a graphical representation of power spectra of local field potentials for Subject 5 according to some embodiments of the present disclosure;

FIG. 10F is a graphical representation of power spectra of local field potentials for Subject 6 according to some embodiments of the present disclosure;

FIG. 10G is a graphical representation of power spectra of local field potentials for Subject 7 according to some embodiments of the present disclosure;

FIG. 10H is a graphical representation of power spectra of local field potentials for Subject 8 according to some embodiments of the present disclosure;

FIG. 10I is a graphical representation of power spectra of local field potentials for Subject 9 according to some embodiments of the present disclosure;

FIG. 11A is a graphical representation of power in the beta frequency range correlated with 130 Hz DLEP power according to some embodiments of the present disclosure;

FIG. 11B is a graphical representation of beta power suppressed by 130 Hz DBS according to some embodiments of the present disclosure;

FIG. 11C is a graphical representation of normalized beta power was according to some embodiments of the present disclosure;

FIG. 12A is a graphical representation of phase-amplitude coupling analysis for Subject 1 according to some embodiments of the present disclosure;

FIG. 12B is a graphical representation of phase-amplitude coupling analysis for Subject 2 according to some embodiments of the present disclosure;

FIG. 12C is a graphical representation of phase-amplitude coupling analysis for Subject 3 according to some embodiments of the present disclosure;

FIG. 12D is a graphical representation of phase-amplitude coupling analysis for Subject 4 according to some embodiments of the present disclosure;

FIG. 12E is a graphical representation of phase-amplitude coupling analysis for Subject 5 according to some embodiments of the present disclosure;

FIG. 12F is a graphical representation of phase-amplitude coupling analysis for Subject 6 according to some embodiments of the present disclosure;

FIG. 12G is a graphical representation of phase-amplitude coupling analysis for Subject 7 according to some embodiments of the present disclosure;

FIG. 12H is a graphical representation of phase-amplitude coupling analysis for Subject 8 according to some embodiments of the present disclosure;

FIG. 12I is a graphical representation of phase-amplitude coupling analysis for Subject 9 according to some embodiments of the present disclosure;

FIG. 13A is a graphical representation of increased HFO oscillation amplitude near the beta rhythm peak according to some embodiments of the present disclosure;

FIG. 13B is a graphical representation showing increased HFO amplitude near beta rhythm troughs according to some embodiments of the present disclosure;

FIG. 13C is a graphical representation showing mean preferred phase of HFOs across subjects according to some embodiments of the present disclosure;

FIG. 14 is a block diagram of the handheld stimulation and recording device of FIG. 1 according to some embodiments of the present disclosure;

FIG. 15 is a block diagram of the computing device of FIG. 2 according to some embodiments of the present disclosure; and

FIG. 16 is a block diagram of the implantable stimulation and recording device of FIG. 3 according to some embodiments of the present disclosure.

### DETAILED DESCRIPTION

The present disclosure is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

Throughout this specification, like reference numbers signify the same elements throughout the description of the figures.

When elements are referred to as being “connected” or “coupled”, the elements can be directly connected or coupled together or one or more intervening elements may also be present. In contrast, when elements are referred to as being “directly connected” or “directly coupled,” there are no intervening elements present.

The subject matter may be embodied as devices, systems, methods, and/or computer program products. Accordingly, some or all of the subject matter may be embodied in hardware and/or in software (including firmware, resident software, micro-code, state machines, gate arrays, etc.) Furthermore, the subject matter may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be for example, but not limited to, an electronic, magnetic, optical, electromagnetic, infrared, or semiconductor system, apparatus, device, or propagation medium. By way of example, and not limitation, computer-readable media may comprise computer storage media and communication media.

Computer storage media is non-transitory and includes volatile and nonvolatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage components, or any other medium which can be used to store the desired information and may be accessed by an instruction execution system. Note that the computer-usable or computer-readable medium can be paper or other suitable medium upon which the program is printed, as the program can be electronically captured via, for instance, optical scanning of the paper or other suitable medium, then compiled, interpreted, of otherwise processed in a suitable manner, if necessary, and then stored in a computer memory.

Communication media typically embodies computer-readable instructions, data structures, program modules or other data in a modulated data signal such as a carrier wave or other transport mechanism and includes any information delivery media. The term “modulated data signal” can be defined as a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above-mentioned should also be included within the scope of computer-readable media.

When the subject matter is embodied in the general context of computer-executable instructions, the embodiment may comprise program modules, executed by one or more systems, computers, or other devices. Generally, program modules include routines, programs, objects, components, data structures, and the like, that perform particular tasks or implement particular abstract data types. Typically, the functionality of the program modules may be combined or distributed as desired in various embodiments.

Recitation of ranges of values herein are merely intended to serve as a shorthand method of referring individually to each separate value falling within the range, unless otherwise indicated herein, and each separate value is incorporated into the specification as if it were individually recited herein. Therefore, any given numerical range shall include whole and fractions of numbers within the range. For example, the range “1 to 10” shall be interpreted to specifically include whole numbers between 1 and 10 (e.g., 1, 2, 3, . . . 9) and non-whole numbers (e.g., 1.1, 1.2, . . . 1.9).

Although process (or method) steps may be described or claimed in a particular sequential order, such processes may be configured to work in different orders. In other words, any sequence or order of steps that may be explicitly described or claimed does not necessarily indicate a requirement that the steps be performed in that order unless specifically indicated. Further, some steps may be performed simultaneously despite being described or implied as occurring non-simultaneously because one step is described after the other step)) unless specifically indicated. Where a process is described in an embodiment the process may operate without any user intervention.

Those skilled in the art will recognize improvements and modifications to the embodiments of the present disclosure. All such improvements and modifications are considered within the scope of the concepts disclosed herein and the claims that follow.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

This disclosure describes the use of evoked potentials recorded from the deep brain stimulation (DBS) lead as a bio-feedback signal to indicate lead/contact placement relative to brain targets and effective stimulation parameters.

It is shown that DBS local evoked potentials (DLEPs) show distinct characteristics depending on contact location relative to the subthalamic nucleus. Therefore, a system is disclosed that uses the DLEP signal to guide or verify electrode placement or select stimulation parameters. Subthalamic DLEP is provided as an example case, but this disclosure applies to other brain targets including the globus pallidus and thalamus. DLEP from the thalamus and subthalamic nucleus has already been recorded.

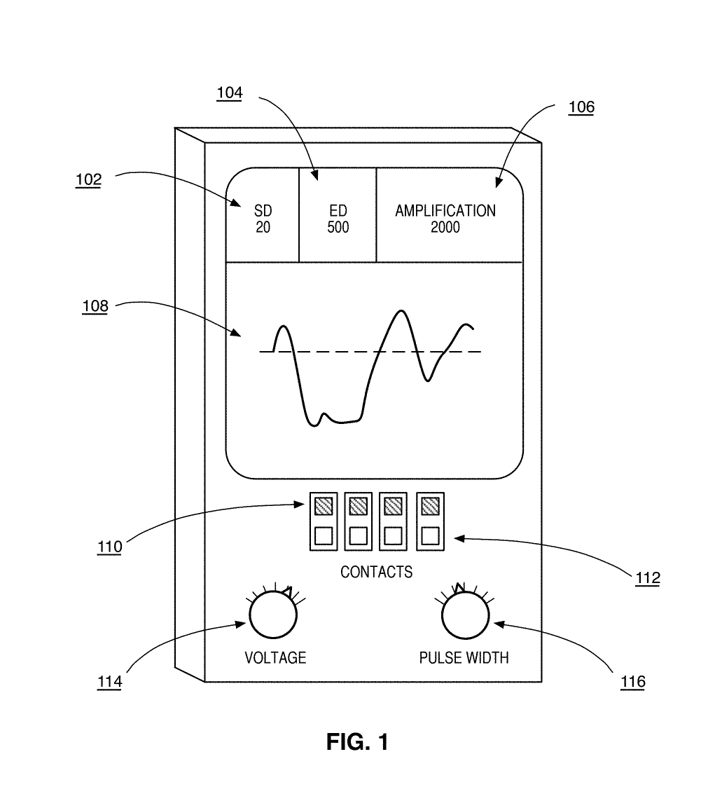

Provided herein is a small handheld device specifically built for delivering stimulation and recordings DLEPs. This device would be used intraoperatively by clinicians to verify electrode placement and effective stimulation parameters. The device would be battery powered and allow the clinician to make changes to the stimulation parameters. The device would also have electronics to blank, amplify, process, and visualize DLEP signals in real-time as stimulation parameters and/or lead location changes. The preferred embodiment of this disclosure would include a color, touch screen display that would allow for changes of the stimulation or recording parameters. Further, a shielded cable would connect the device to the DBS lead to mitigate the risk of introducing extrinsic noise. FIG. 1 shows a schematic of this design.

Referring now to FIG. 1, a graphical depiction of a hand held device to deliver stimulation and record DLEPs in the intraoperative environment according to some embodiments of the present disclosure is shown. The device operable to provide an ability to select between stimulation and recording contacts 110 112; adjust stimulation and recording parameters 102 (start blanking delay) 104 (end blanking delay) 106 (stimulation amplification) 114 (pulse voltage) 116 (pulse width); process the DLEP signal; and visualize the DLEP recordings in real-time 108. A clinical operator would operate this device to verify electrode placement and effective contacts both electrophysiologically and behaviorally. The device is operable to control blanking delays prior to a pulse 102, control blanking delays after a pulse 104, and stimulation pulse amplitude 106.

Also provided herein is a combined system for recording all physiological signals during electrode placement. The system could be used to record single-unit activity before DBS lead placement and DLEPs after DBS lead placement. In the preferred embodiment, the system would include a cart with signal processing hardware and a computer for real-time processing and data visualization, and a shielded cable would connect the system to the subject's DBS lead.

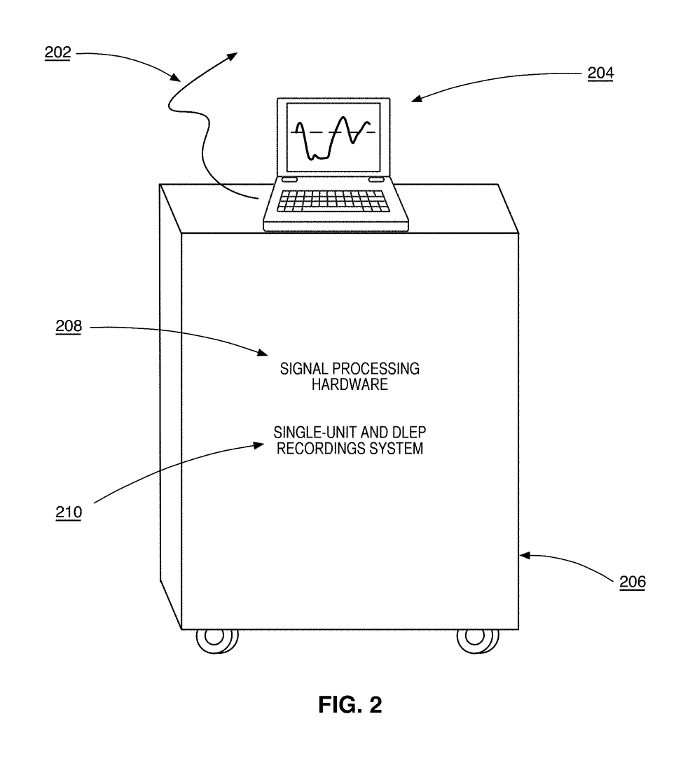

The DLEP-related hardware would include stimulation generating hardware, amplifiers with blanking tied to stimulation pulse timing, anti-parallel diode clamps to reduce artifact amplitude, and hardware to change contacts used for stimulation and recording. A schematic of this design is shown in FIG. 2.

Referring now to FIG. 2, a system for recording neural signals related to DBS lead implantation according to some embodiments of the present disclosure is shown. The system operates to record single-units and DLEPs 210, and it will include a console with stimulation and recordings hardware 202 208 and a computer for real-time data processing and visualization 204.

Also provided herein is a system including DLEP recording capabilities within the implantable pulse generator (IPG). The device would have integrated systems for signal amplification and blanking, time-aligning and averaging, DLEP quantification and interpretation, real-time control of stimulation parameters, and/or communication of DLEP data to a wireless receiver. With these capabilities, the IPG could operate in closed-loop mode were all stimulation parameter selection and adjustments are carried out automatically after assessing DLEP signals, either periodically or continuously. In the preferred embodiment, the amplifiers and blanking subsystem is capable of self-regulating the level of amplification and blanking timing so that the amplifiers do not saturate. The subsystem would also be capable of sending error messages if appropriate DLEP signals could not be recorded.

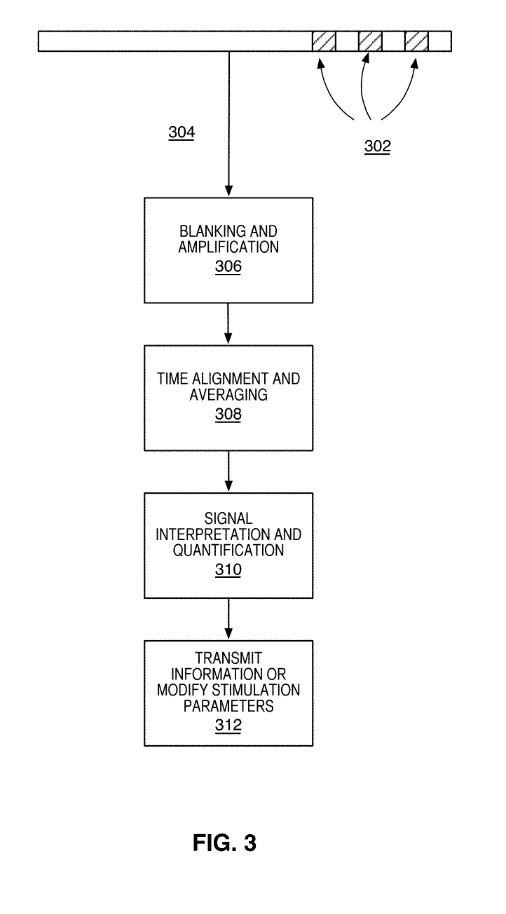

Referring now to FIG. 3, a block diagram schematic of DLEP recording capabilities included in an implantable pulse generator according to some embodiments of the present disclosure is shown. Electrical responses 304 are recorded in reaction to the stimulus transmitted via the electrodes 302. Blanking and amplification 306 is performed to remove artifacts and increase amplifier gain and stability during the stimulation process. The resultant recorded signal is time aligned and averaged 308 to produce an aggregate signal that better represents a normalized response. The aggregate signal is analyzed and quantified 310 to produce a parameterized representation of the aggregate signal. The process may be repeated by adjusting 312 the recording setup (electrode placement or stimulation parameters), or the parameters may be transmitted and/or stored for clinical use.

Also provided herein is an application that would pair with DLEP recording technology and be used by clinicians or patients for monitoring contact placement or stimulation parameters. In one embodiment of this disclosure, the “app” could pair with DLEP monitoring capabilities in the IPG and inform selection of stimulation parameters by the patient or clinician in follow up appointments after the DBS lead implant. In another embodiment, the DLEP recording setup could be modular, with sterile hardware in the surgical field and wireless transmission to a device with the “app” for visualization, analysis, and interpretation.

In all embodiments of the disclosure described above, the actual DLEP signal may or may not be displayed. In both cases, an interpretation of the DLEP signal and its implications for contact location and stimulation parameters will be included.

The following examples are provided by way of illustration and not limitation.

### EXAMPLES

### Subthalamic Evoked Potentials Reflect Functional Connectivity of Human Basal Ganglia

Evoked potentials can reveal important characteristics of neural circuits, and experimentation recorded evoked potentials during deep brain stimulation (DBS) of the subthalamic nucleus (STN) in humans with Parkinson's disease. The DBS local evoked potentials (DLEPs) were highly stereotyped across subjects, and a 3-dimensional biophysical model of the subthalamic nucleus, globus pallidus, and hyperdirect pathways revealed that the evoked potentials were the result of complex interaction between excitatory and inhibitory synaptic currents in the STN. DLEP signals possessed time- and frequency-dependent properties, and were related to pathological beta band oscillations, exaggerated phase-amplitude coupling, and electrode contact locations. DLEPs are a novel signal that reveals functional connectivity of the human basal ganglia and can be used to guide or verify DBS lead placement, probe the pathological basal ganglia, and elucidate the mechanisms of DBS.

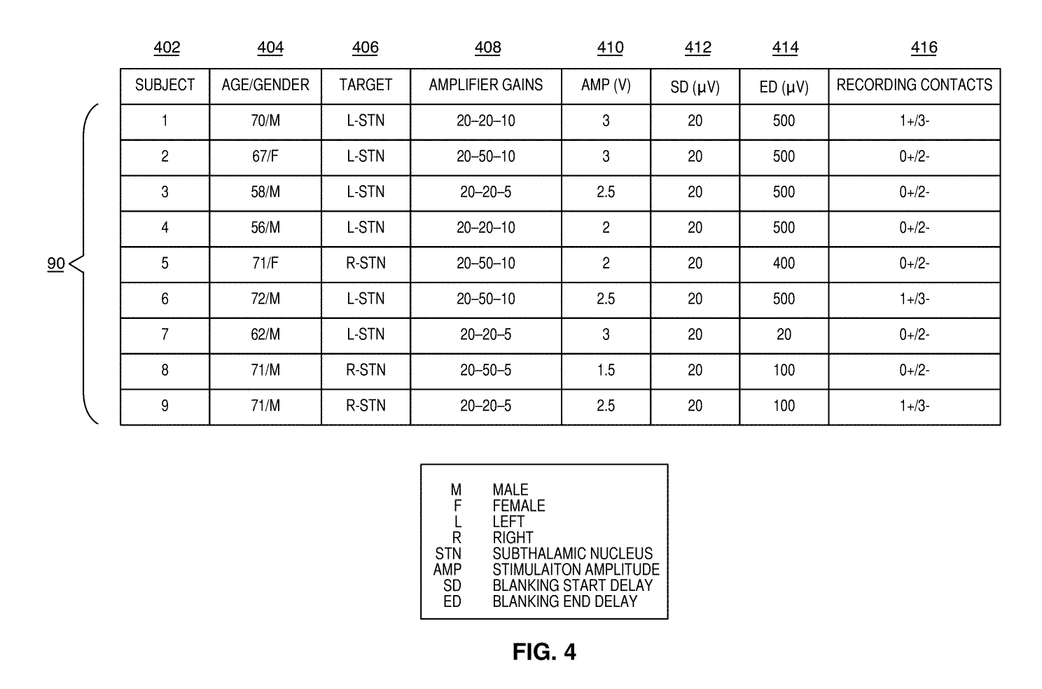

As provided herein, STN DLEP characteristics were quantified in subjects with PD and developed a computational model of the STN and globus pallidus externus (GPe) was developed to determine the neural origins of the DLEP signals. Further, the relationship was characterized between DLEPs, local field potentials (LFPs), and electrode location in the human STN. Persons with PD undergoing DBS lead implant in the STN were recruited to participate in the study. The Institutional Review Board at Duke University approved the study protocol, and subjects participated on a volunteer basis following written informed consent. Twelve subjects were consented for the study, but three withdrew prior to any study procedures, and nine subjects were included in the analysis (FIG. 4). Polyphasic DLEPs were highly stereotyped across subjects, and exhibited both time-dependent and DBS frequency-dependent properties. FIG. 4 comprises information related to the subject 402, the age and gender of the subject 406, the amplifier gain (specified per stage) 408, stimulation pulse gain 410, blanking prior to pulse 412, blanking after pulse 414 and recording contact 416. This information is specified for the nine participating subjects 90.

### Short Latency DLEPs

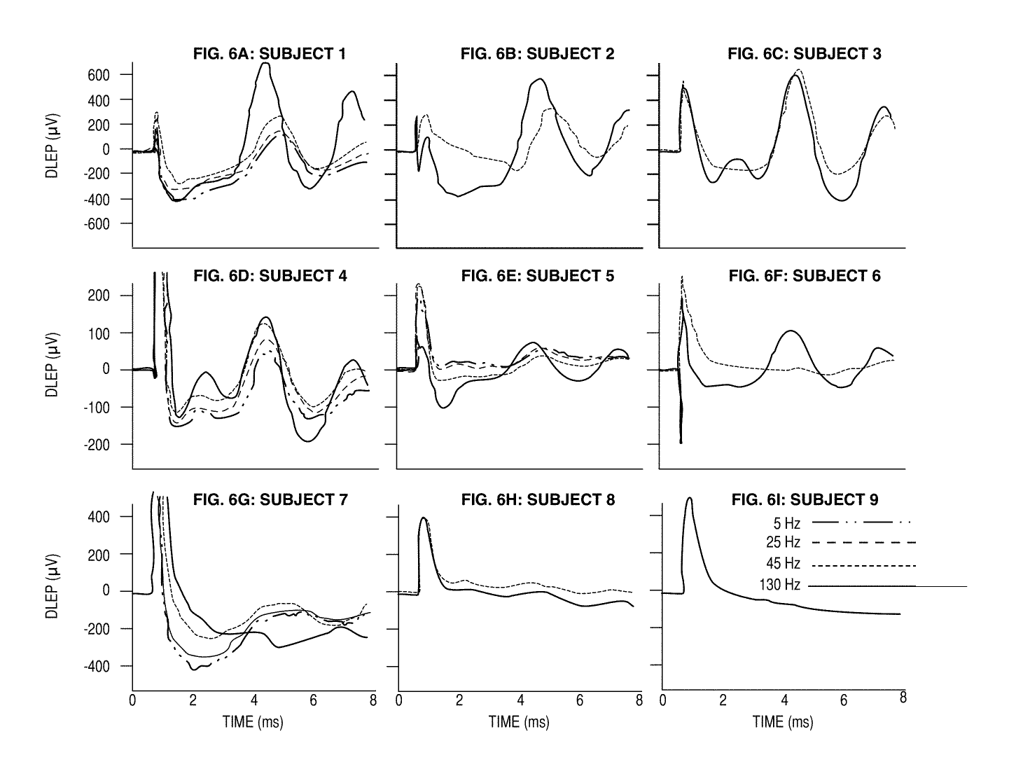

The average short latency (0-7.7 ms) DLEPs for the time interval from 5-15 s after the start of stimulation are shown in FIG. 6. The DLEPs were characterized by an early positive deflection (˜0.7-1.5 ms), an early negative deflection (˜1.5-3.0 ms), a late positive deflection (P1, ˜4.0-5.5), a late negative deflection (N1, ˜5.5-6.5 ms), and a second late positive deflection (˜6.5-7.7 ms). To demonstrate that the multiphasic DLEPs were indeed physiological, and not an electical artifact, DBS were delivered with pulses that alternated between anodic- and cathodic-phase-first in one subject (subject 5). There were no differences in DLEPs between the two pulse polarities, and the average DLEP across all pulses was calculated (FIGS. 6 & 7, middle panel). Time- and frequency-dependent changes in the DLEP response (see below) also imply a dynamic physiological response and not a stereotyped artifact.

The characteristics of the DLEPs were dependent on DBS frequency. First, the amplitude of P1 was greater for higher DBS frequencies during this time interval (c.f., time-dependent changes below). Second, the DLEP elicited by high frequency stimulation (130 Hz) exhibited deflections in the early negative phase that were consistent with being continuations of the multiphasic DLEP signal evoked by the previous DBS pulse (see subjects 3,4,5). This effect became less noticeable late in the stimulation epoch.

The DLEPs recorded in Subjects 7-9 were not consistent with those from the remainder of the subjects. All three subjects lacked late positive deflections, although evidence of early and late negative deflections was observed in subjects 7 and 8. Subject 6 lacked P1 deflections during 45 Hz stimulation, but P1 emerged over the course of the first 100 ms of 130 Hz stimulation (see time-dependent changes below). Subject 9 did not have any of the late DLEP characteristics shared by the other subjects.

### Long Latency DLEPs

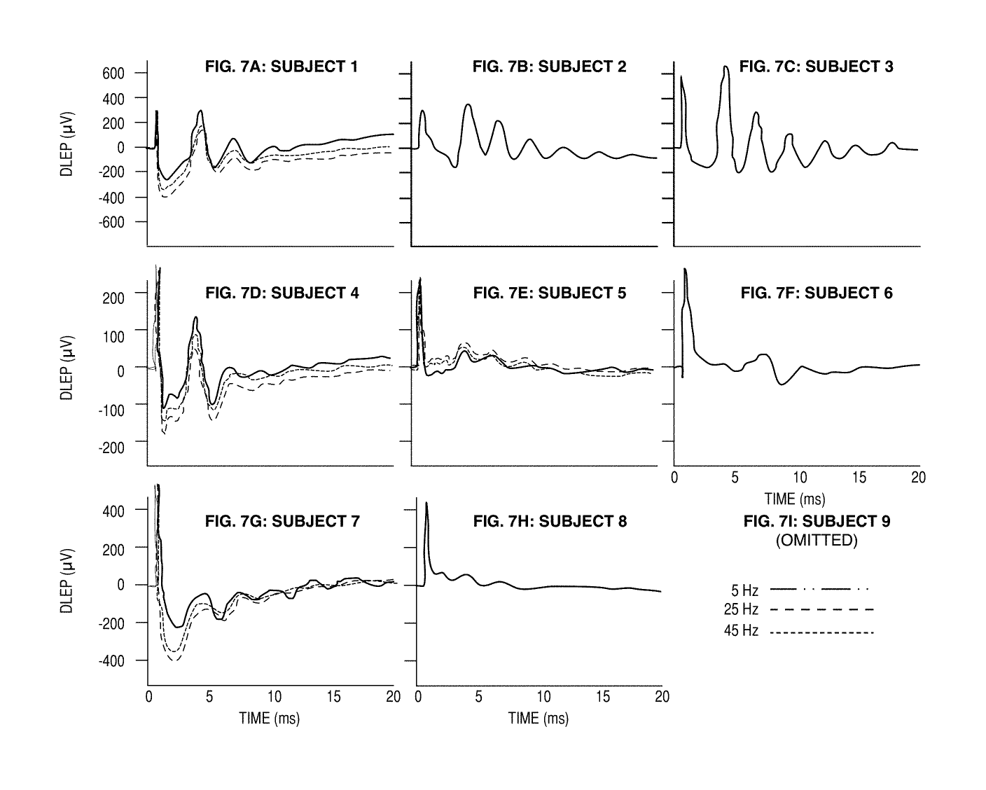

The average long latency (0-22 ms) DLEPs for the 5-15 s interval are shown in FIG. 7. Only DBS frequencies ≤45 Hz were included because those frequencies had sufficiently long interpulse intervals to visualize long latency responses. The long latency DLEPs revealed that the quasi-periodic oscillations persisted well after the stimulation pulse. There were up to six late positive and negative deflections within the 22 ms interval (see subjects 2 and 3). The amplitude of the positive and negative deflections diminished during the interval, but the relative timing between the positive deflections remained relatively constant (˜3 ms), only slowing slightly near the end of the interval. Therefore, the frequency of the oscillations after each DBS pulse was ˜333 Hz, which is similar to the frequency of high frequency oscillations recorded in STN and to the modulated frequencies observed in STN phase-amplitude coupling. DLEPs were similar across measurements during different frequencies of DBS, and higher frequencies tended to produce higher amplitude positive deflections, and longer lasting oscillations.

### Simulated DLEPs

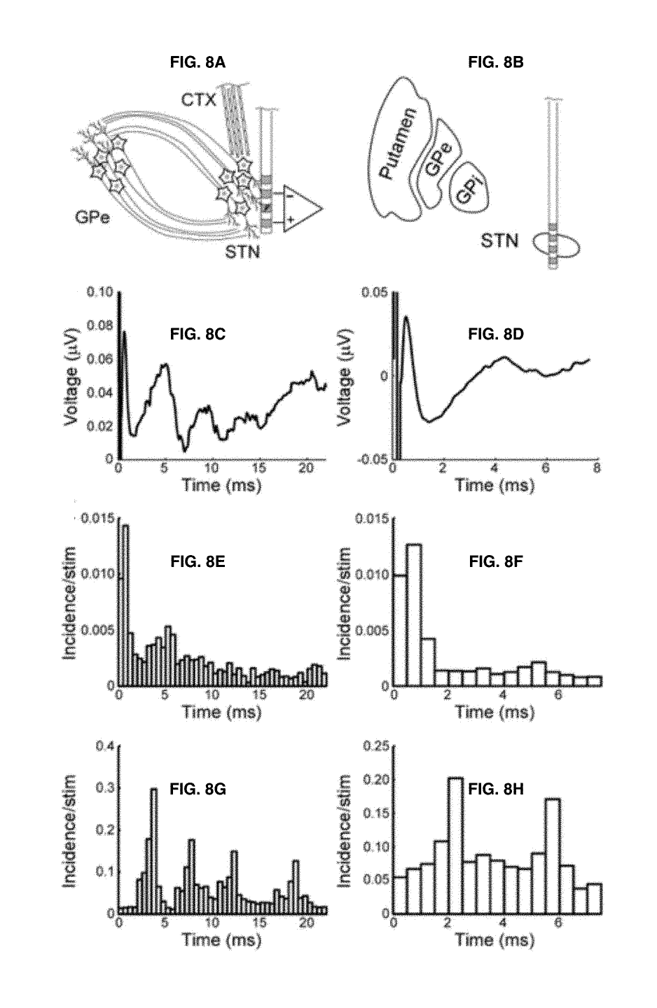

Disclosed herein is a 3-dimensional biophysical model of STN DBS in the STN-GPe subcircuit to investigate the neural origin of DLEPs (FIG. 8). Model-generated DLEPs for 45 Hz and 130 Hz DBS were remarkably similar to DLEPs from humans with PD indicating that the modeled STN-GPe subcircuit and the afferent cortico-STN axons were sufficient to generate DLEPs (830 840). All phases of the human DLEP were present in the model DLEP, however there were fewer positive deflections in the modeled 45 Hz DLEP compared to clinical DLEPs of some subjects. STN neurons were relatively quiescent in the interpulse intervals due to high inhibitory tone from the highly active GPe neurons (850 860). This is consistent with reports that STN neurons are silenced or exhibit lower firing rates during high frequency stimulation. The probability of model STN neuron firing increased 3-5 ms after a DBS pulse, which matched the timing of the late positive deflection (P1) and is consistent with reports of STN action potentials during high frequency DBS interpulse intervals. GPe neurons were excited by STN axons, which were directly excited by DBS. Due to the highly synchronized input and the recurrent inhibition within GPe, GPe neurons tended to fire periodically at intervals of 3-4 ms (870 880). Indeed, similar periodic firing is observed in GPe single unit recordings in parkinsonian monkeys during STN DBS. Therefore, the periodic pallidal firing is responsible for the periodic late DLEP phases. Temporal summation of excitatory synaptic currents in GPe during high frequency stimulation led to GPe action potentials earlier in the interpulse interval (880), and temporal summation may also be responsible for the shorter latency of P1 and N1 observed in the early clinical 130 Hz DLEP.

### Time-Dependent Changes in 130 Hz DLEPs

130 Hz DBS caused time-dependent changes in the experimental DLEP during stimulation, and these changes quantified using the l²-norm of the difference vector between individual DLEPs and the average DLEP (910). During the first 100 ms of stimulation (910, inset), the magnitude of P1 increased rapidly with successive pulses, and the P1 latency shortened (820). Interestingly, P1 produced by the first 130 Hz DBS pulse in subject 6 was non-existent, similar to the 45 Hz DBS DLEP, but it emerged and grew in amplitude over the first 100 ms of stimulation. The magnitudes and latencies of P1 and N1 exhibited slow time-dependent changes (930 940 950 960). After a brief increase in magnitude, P1 and N1 magnitudes decayed during the stimulation epoch, and the normalized P1 amplitude was significantly lower for the 50-60 s interval compared to the 5-15 s stimulation interval across subjects (paired t-test, p=0.007, n=5; 970). Latencies for each deflection increased in parallel between 5-15 s and 50-60 s (p=0.0279; mean diff.=0.3 ms; n=5; 980). For subjects receiving 300 s of stimulation, magnitudes and latencies stabilized by —100 s (950 & 960). The normalized P1 amplitude and N1 latency continued to decrease and increase, respectively, in subjects that received 300 s of stimulation.

### Local Field Potential Spectral Estimates

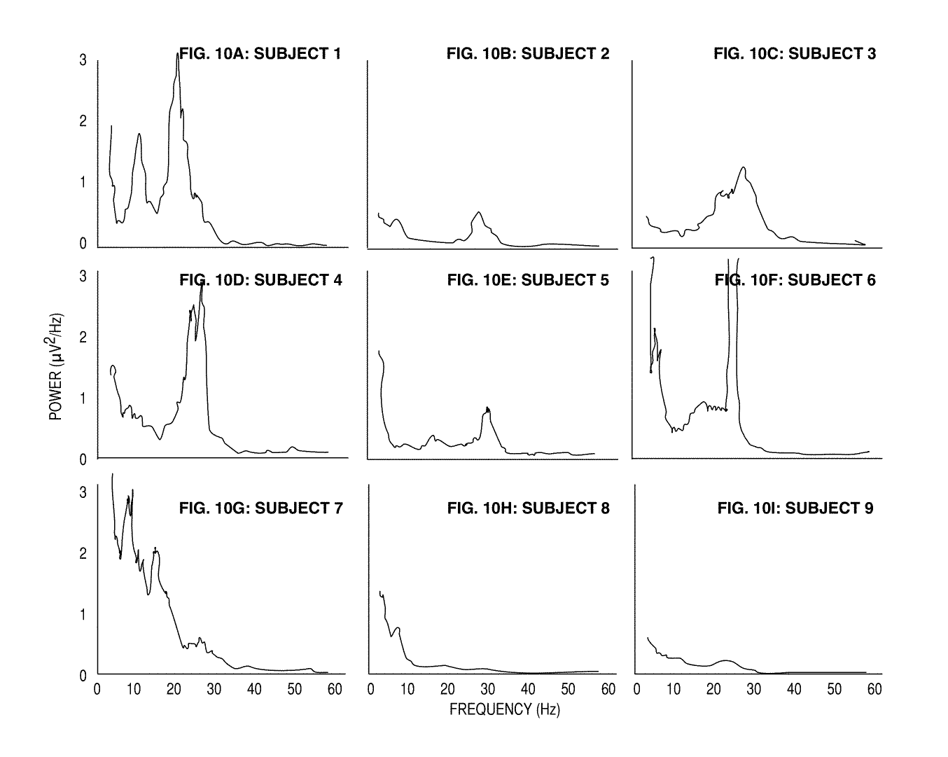

LFP recordings revealed that robust DLEP responses were predictive of beta frequency oscillations in the STN of patients with PD. Distinct peaks in the beta frequency range (10-35 Hz) were observed in most subjects, and subjects lacking beta peaks also exhibited poor DLEP signals (FIG. 10). Indeed, beta band power was correlated with DLEP power during 130 Hz DBS (1110), suggesting that contact location was an important determinant for both signals. DBS frequencies differentially suppressed beta band power (F=6.00, p=0.004), and 130 Hz DBS significantly suppressed beta band power relative to 5 Hz, 20 Hz, and 45 Hz DBS (p<0.05, 1130), but the beta suppression did not vary meaningfully with time—in contrast to the time-dependent changes in the DLEPs (1120).

### Phase-Amplitude Coupling

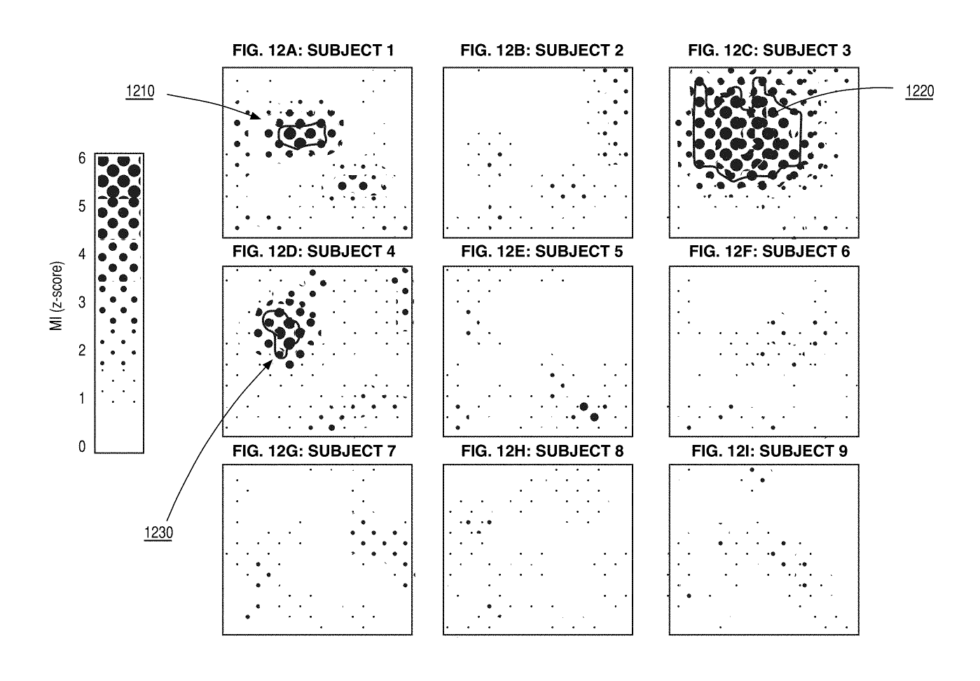

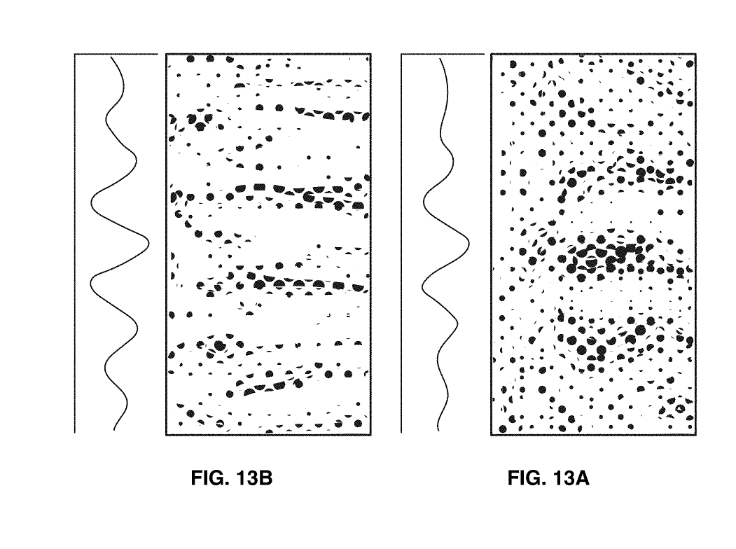

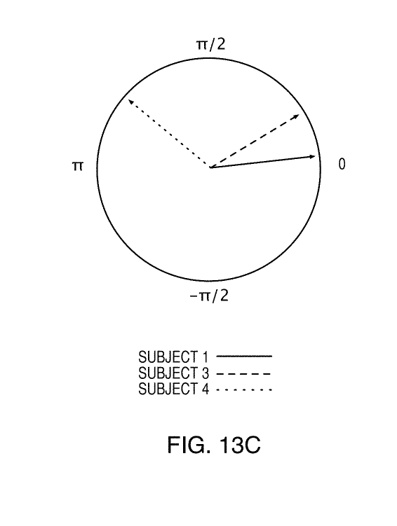

Cross-frequency coupling is observed in the STN of patients with PD wherein the amplitude of high frequency oscillations (HFOs, 250-350 Hz) is modulated by the phase of the beta frequency oscillatory activity. The similarity of the frequencies of the DLEP oscillations and the HFOs coupled to the beta rhythm, prompted investigation of phase-amplitude coupling (PAC) in the recordings. PAC was observed in three subjects (FIG. 12). These subjects (1, 3, & 4) were a subset of those with distinct P1 phases in the DLEP, suggesting that robust DLEPs may be necessary, but not sufficient for cross-frequency coupling. The preferred phase of the HFOs on the modulating beta rhythm was examined in the three subjects with significant PAC. HFOs in subjects 1 and 3 occurred preferentially near the β peaks, but in subject 4 occurred preferentially near the β troughs (FIG. 13). Note that recorded signals for subject 4 were not inverted before performing PAC analysis as they were for the DLEP analysis. HFO coupling to both β peaks and troughs is reported, and the data indicate that the coupling is dependent on the bipolar recording configuration and the location of the recording contacts relative to the borders of the STN. Further, these data support that some subjects had DLEPs that were inverted versions of DLEPs in other subjects, and because of the bipolar recording, contact location is an important determinant for DLEP and LFP polarity.

### Contact Lead Locations

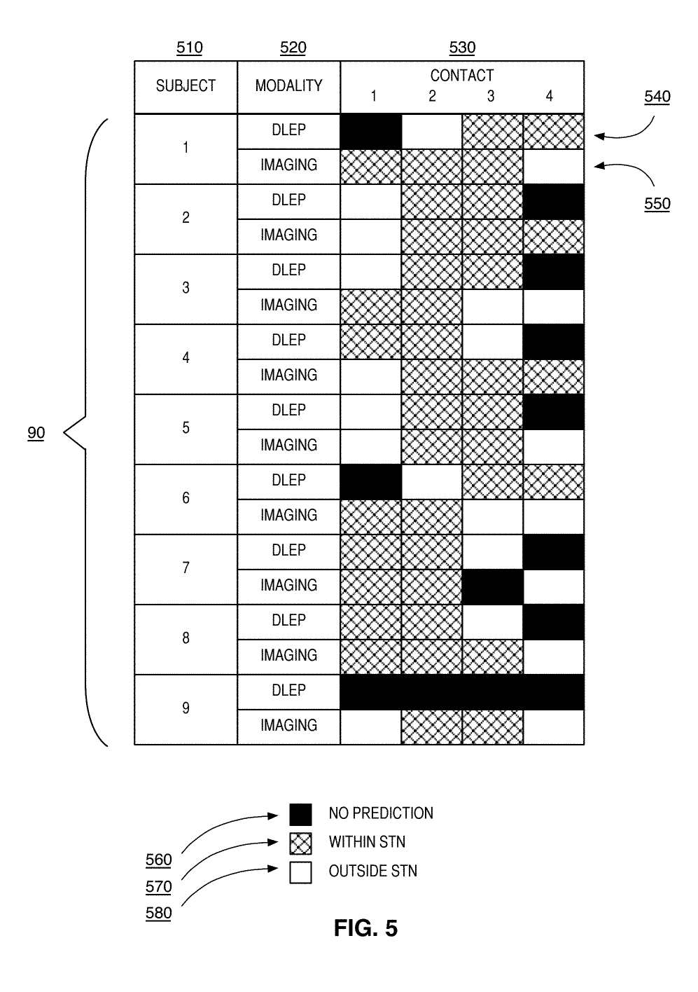

Collectively, the DLEP, computational modeling, LFP power spectra, and PAC data suggest that contact location may determine the character of the DLEPs. To investigate the relationship between DLEPs and contact location, a set of blinded predictions about contact locations based on the recorded DLEPs was compiled and compared these predictions to contact locations based on post-operative imaging analysis. It was predicted that the stimulating contact was within STN for all subjects with DLEP signals, since contacts outside STN would likely be unable to elicit the stereotyped DLEP response. It was predicted that subjects with non-inverted DLEP signals had the negative recording contact within the STN, while subjects with an inverted DLEP signals (4, 7, 8) had the positive recording contact within the STN. The predictions were also informed by single unit recordings showing inhibition of STN neurons approximately 6-7 ms after a DBS pulse, which corresponded to the N1 phase observed in the DLEP signal. Inhibitory currents would be seen as sources by an extracellular recording electrode, and therefore observed as positive deflections when recorded from a positive recording contact or negative deflections when recorded from a negative recording contact. Two investigators blinded to the DLEP-based predictions performed the post hoc imaging analysis. Since the STN cannot be visualized via computed tomography (CT), pre-operative magnetic resonance (MR) and post-operative CT images were merged to estimate contact locations relative to the boundaries of the STN. Contact locations predicted from the DLEP analysis matched the imaging-based contact locations for 11/16 contacts (˜70%) predicted to be within STN (FIG. 5). Predictions were unable to be made regarding contact locations for subject 9 due to lack of DLEP signal. The information reflected in FIG. 5 includes the subject 402, modality 520, and the contact 530. The modality includes both DLEP predictions 540 and imaging predictions 550. Predictions values include no prediction 560, within STN 570, and outside STN 580. This information is specified for the nine participating subjects 90.

STN DLEPs exhibit a distinctive, multiphasic signature that reflects functional connectivity between STN and GPe. DLEPs exhibited frequency- and time-dependent characteristics and were related through contact location to beta frequency oscillations and exaggerated coupling between the beta rhythm and the amplitude of HFOs. Therefore, DLEPs reveal important insights into the mechanisms of DBS and may serve as a tool for investigating basal ganglia subcircuits and guiding DBS lead implant location.

### Origin of the DLEP Signal and Functional Connectivity of the STN

It is well established that STN receives afferent input from the pallidum through the indirect pathway and from the cortex via the hyperdirect pathway, and DLEPs reflect the interaction of these two inputs following local stimulation. The early positive phase of the DLEP resulted from direct and short latency antidromic excitation of STN neurons, and the early negative phase reflected strong proximal inhibition of STN neurons evoked by activation of the pallidal terminals. Axons presynaptic to excitatory synapses were also directly activated by DBS, but their effects on STN firing were observed later in the interpulse interval due to the distal distribution of excitatory synapses on STN neurons. High frequency oscillations caused by quasi-periodic pallidal inhibition of STN with an interval of approximately 3-4 ms were the most distinctive feature of the DLEPs. These oscillations are consistent with single-unit recordings in parkinsonian primates and humans. Pallidal neurons fire periodically with 3-4 ms intervals in response to STN DBS. Further, STN single unit recordings reveal action potentials in the interpulse interval with latencies that match the positive deflections of the DLEP signal, and inhibition at a latency of approximately 6 ms. Thus, the late DLEP oscillations reflect excitatory synaptic currents and the resulting action potentials interrupted by brief period of inhibition via pallidal afferents. Although DLEPs could reflect membrane currents in afferent axons from the pallidum, this does not account for the variable presence of the late positive phases of the DLEP, and total transmembrane currents for axons—especially myelinated axons—are very small compared to transmembrane currents through dendrites and somata due to large differences in surface area. Alternately, the multiphasic DLEP signal could reflect time-locked bursting of STN neurons after a DBS pulse. Indeed, STN neurons exhibit more burst firing activity in PD. However, intraburst firing rates of STN neurons are too low (<250 Hz) and variable to account for the oscillations, and this does not account for the variable presence of the late positive phases of the DLEP.

There are several limitations to the model used to interpret the experimental DLEPs. First, the model did not include slower synapses, such as NMDA or metabotropic receptors, because the observed evoked responses were fast and unlikely to be influenced greatly by slower synaptic dynamics. Second, the model did not include plasticity and did not capture the changes in DLEPs that occurred over the course of minutes of stimulation. Third, both cell types observed in the GPe were not included, and model pallidal neurons were based on the more numerous type I pallidal neurons. Finally, finite element modeling and 3-dimensional DBS lead representations could further enhance the model's accuracy, but are unlikely to change the qualitative description of the DLEP signal.

### Time Course of DLEP Evolution

Changes in DLEP signals during high frequency DBS reflect physiological changes that may contribute to DBS efficacy. The increase in magnitude of the P1 peak over the first 100 ms likely reflected temporal summation of excitatory synaptic currents resulting in increased excitation in the STN. The amplitude of the P1 peak then decreased substantially during stimulation, which may reflect synaptic depletion of afferent excitatory synapses in the STN. Similar reductions in synaptic strength have been observed during high frequency thalamic DBS in rat brain slices. The P1 and N1 latencies gradually increased in parallel during stimulation, indicating that both latencies are determined by a single underlying mechanism—the latency of the pallidal inhibition that is evoked by excitation of the STN efferents with each DBS pulse. This would imply that the latency of orthodromically driven action potentials in the pallidum increases over time during high frequency STN DBS. Indeed, this has been observed in parkinsonian primates, and the time course of the increase in latency matched exquisitely the increase in P1 and N1 latency observed here. Therefore, changes in P1 and N1 latency likely reflect changes in pallidal excitation latency and not local changes within STN.

### Relationship to HFOs

Previous recordings revealed the presence of HFOs (250-350 Hz) and significant PAC in the STN. The observed PAC occurs when HFO amplitude is modulated by the phase of beta frequency oscillations. The oscillations in the DLEP (see FIG. 7) occur in the same frequency range as the HFOs observed in spectral and PAC analyses, and significant PAC was only observed in the subset of subjects with robust DLEP responses, suggesting that these signals may be mechanistically related.

HFOs and STN single-unit spikes are anti-phase coupled to the beta modulating rhythm. This implies that HFOs are preferentially expressed during the phase of the beta rhythm reflecting STN inhibition. The STN receives strong inhibition from the GPe, suggesting that GPe afferents could be the driver for HFOs observed in the STN, and the results strongly support this hypothesis. It is proposed that synchronous STN to GPe excitation during the exaggerated beta rhythm causes strong firing in the GPe, which is synchronized at high frequencies by intrapallidal inhibition. This high frequency synchronous pallidal firing is then reflected in the inhibitory phase of the STN beta rhythm.

### Imaging Analysis Limitations

The DLEPs, LFPs, and modeling analysis all suggested that contact location was an important determinant for the presence and characteristics of DLEP signals. DLEP power was correlated with beta band power, and beta band oscillations are prominent within STN. Computational modeling revealed the subthalamic neural origin of the DLEP signal and showed that non-inverted DLEPs were consistent with placement of the negative recording contact within the STN. These analyses were consistent with single-unit activity in non-human primates and humans. Predictions were developed regarding contact locations based on the recorded signals and compared these predictions to contact locations based on blinded image analysis. Imaging analysis was performed by merging post-operative CT and pre-operative MR images since the STN cannot be visualized on CT scans. While this is currently the preferred method for assessing contact locations post-operatively, there are drawbacks to this approach. The brain can shift between scans, which reduces the accuracy of the contact location estimates relative to STN. This problem is exacerbated and perhaps caused by subdural air invasion and CSF outflow after craniotomy and subsequent dural breach. Further, the resolution of the CT and MR images—and therefore the variance of the estimate—is on the same order of magnitude as the distance between contacts on the DBS lead. The fact that MRI-based targeting is supplemented by intraoperative single-unit recordings is another indicator of uncertainty associated with neuroimaging-based STN localization. Given these limitations, it is not surprising that there were some discrepancies between imaging- and DLEP-based contact location predictions.

### Implications for DBS Therapy

DLEPS are a novel and potentially important tool for assessing DBS contact location within STN. Accurate and easily accessible contact location data would greatly assist with DBS parameter programming and intra-operative STN localization. This could reduce time and costs associated with surgery and follow-up programming visits, and it could positively affect patients with PD since lead misplacement is a leading cause of suboptimal outcome after DBS implant.

DLEPs contribute three important elements to understanding of the mechanisms of DBS. First, they strongly support that STN efferents are excited by DBS, and that STN somata and axons can be functionally decoupled during stimulation. Second, the effects of stimulating the efferent STN axons can be observed via the returning inhibition from GPe after several minutes of DBS, indicating that STN is not effectively silenced by DBS (i.e., its axons are influencing GPe and afferent axons are still affecting STN). Third, the decrease in amplitude of the P1 peaks with a time course similar to the amelioration of some of the axial symptoms of PD by DBS suggests that synaptic depletion, particularly for excitatory synapses, should be further investigated.

Further, LFP recordings during low and high frequency DBS also increase understanding of the mechanisms of DBS. LFP recordings during low frequency DBS are rarely reported because most recording methods use low pass filters to exploit the separation in the frequency domain between the physiological signal and high frequency stimulation artifact. Further, as demonstrated here, LFPs may be corrupted by short latency evoked activity in the STN. A combination of amplifier blanking, linear interpolation, and template subtraction was provided to overcome these challenges and demonstrate stimulation frequency-dependent suppression of beta oscillatory activity. This DBS frequency-dependence paralleled suppression of symptoms, and, indeed, suppression of abnormal synchronous oscillations is a proposed mechanism of effective DBS. However, beta band activity was not present in all subjects, possibly due to differences in electrode position, which challenges the notion the beta band activity alone can serve as a feedback signal for closed-loop DBS.

There is some disagreement whether stimulation at high frequencies is exclusive in its ability to produce time-locked responses in the basal ganglia. In rat STN slices, high frequency but not low frequency stimulation was capable of producing only stimulation-locked responses, and in awake, freely moving rats stimulation-locked effects were more potent at downstream nuclei for high frequency stimulation. However, pallidal single-unit recordings during pallidal microstimulation produced time-locked responses at both low and high stimulation frequencies. The results provide further evidence that low frequency stimulation can produce a complex and long-lasting time-locked response in STN. Therefore, the therapeutic mechanism of DBS seems unlikely to hinge on stimulation's ability to produce time-locked responses in the STN. DLEPs also provide evidence against chaotic desynchronization as a mechanism of DBS. In the chaotic desynchronization model, high frequency subthalamic stimulation desynchronizes firing of pallidal neurons. However, the current results—and previous single unit recordings in the pallidum—suggest that pallidal neuron firing is likely highly synchronized to DBS pulses across stimulation frequencies.

Finally, DLEPs are a candidate biomarker for closed-loop DBS. DLEPs are a new biomarker that not only can probe the local neural circuits, but can also inform electrode placement and the selection of stimulation contacts and parameters. This is supported by the observation that effective stimulation parameters were exclusively able to produce inhibition in the STN with a latency of ˜6 ms after the DBS pulse.

### Methods

### Subject Information

Persons with PD undergoing DBS lead implant in STN were recruited to participate in the study. Subjects withheld PD medications for 12 h prior to surgery, and short-acting sedation was given to subjects prior to research activities as part of routine medical care. Subjects were assigned to one of two groups based on the presence of tremor during preoperative care. Subjects with tremor had 60 s trials and received four different DBS frequencies (5, 20, 45, 130 Hz) interspersed with DBS-OFF epochs. Subjects without tremor only received 45 Hz and 130 Hz DBS with DBS-OFF epochs intervening, but trials were 300 s long. Trial lengths were chosen because of possible differences in the time course of DBS effects on symptoms of tremor and akinesia/bradykinesia in PD, and the design allowed examination of several DBS frequencies and long epochs of stimulation.

### Intraoperative Protocol

The intraoperative research protocol began after completion of microelectrode recordings, DBS lead placement, and clinical assessment of therapeutic benefit. A sterile connection was made between the DBS lead and the stimulation and recording equipment (described below), and a stimulation return electrode (StimCare Carbon Foam Electrode, Empi) was placed on the chest ipsilateral to stimulation to simulate monopolar stimulation with the DBS implantable pulse generator case as the counter electrode. DBS was presented in alternation with the DBS-OFF condition. Stimulation pulses were symmetric and biphasic with a 90 μs per phase pulse width.

The DBS leads had four cylindrical platinum-iridium contacts (d: 1.27 mm; h: 1.5 mm) separated by 1.5 mm of insulation (Medtronic DBS Lead Model 3387). Stimulation was delivered through one of the two middle contacts at an amplitude sufficient for therapeutic benefit as determined by the attending neurologist, and bipolar recordings were made from the two surrounding contacts. The ventral recording contact always served as the positive input (+) and the dorsal recoding contact served as the negative input (−) to the differential recording, and depending on the location of the recording contacts relative to the borders of the STN the signals could be inverted relative to signals recorded in other subjects. During DBS-OFF, the recordings captured local field potentials; and during DBS the recordings also included DLEPs.

Bipolar differential recordings were made between the two contacts surrounding the stimulating contact, and the lead implant cannula or the burr hole retractor (subject 5) served as the reference for the recorded signal. The recording instrumentation was developed to record evoked potentials in the presence of DBS and has been described in detail elsewhere⁸. Briefly, the signal was passed through three series biopotential amplifiers (Stanford SR560) with diode clamps between each amplification stage to clip stimulation artifact. The second- and third-stage amplifiers were blanked 20 μs before (start delay) and 20-500 μs after (end delay) each stimulation pulse to enable greater signal amplification. The original recording instrumentation was modified slightly by removing the relay at the stimulator that disconnected the stimulating contact in-between pulses. Signals were digitized at 100 kHz (NI USB-6216) and saved for post hoc data processing and analysis.

In subjects with 60 s trials (n=4), four different DBS frequencies were tested in a randomized block design with 3 blocks (5 Hz, 20 Hz, 45 Hz, and 130 Hz). Only one subject competed all three blocks, and there were no observed differences in DLEPs between blocks in subjects that completed more than one block. Therefore, only one block from each subject was analyzed. Subjects were included in the short trial group if observed parkinsonian tremor in preoperative care because the time course of the response of tremor to effective DBS is short—on the order of seconds. Tremor was recorded with an accelerometer in a subset of these subjects, but due to the small sample size and variable presence of tremor in the DBS-OFF condition, tremor measurements were not revealing.

In subjects with 300 s trials (n=5), only two DBS frequencies were tested (45 and 130 Hz), and DBS frequencies were presented in a single block with order randomized across subjects. Due to early withdrawal from the study protocol, subject 2 only received a short 15 s 130 Hz trial, and subject 9 did not receive 45 Hz stimulation. Subjects included in the long trial group did not show prominent tremor, and the trials were appropriate for the longer time course of akinetic symptom response to DBS—on the order of minutes. It was attempted to measure bradykinesia with an alternating finger tapping task on a computer mouse, but only three subjects had complete data sets and the data were not revealing.

### DLEP Signal Processing

Recorded signals were pre-processed by high-pass filtering (3^{rd} order Butterworth, 2 Hz cutoff) to remove offset and slow signal components. For consistency, signals were blanked digitally for 500 μs after each DBS pulse—the longest blanking period used experimentally. Evoked potentials were time aligned and averaged across DBS pulses, and potentials for three subjects (4, 7, & 8) were inverted before plotting and quantification of signal characteristics unless otherwise noted. The amplitudes and latencies of the late positive and negative DLEP deflections (P1 and N1) were quanitified, and paired t-tests were used to assess significant changes in DLEP parameters at different time epochs. The l²-norm of the difference vector between individual and average evoked potentials was quantified to observe whether evoked potentials changed during the course of stimulation. The DLEP signal power in the interpulse interval during 130 Hz DBS was calculated by squaring the signal and integrating across time.

### Power Spectral Estimates

LFP data were collected during DBS-OFF epochs. LFPs were high-pass filtered (2 Hz cutoff; 3^{rd} order Butterworth) to remove any non-zero signal offset. Next, signals were band-pass filtered from 2-100 Hz and down-sampled to 400 Hz before spectral estimation. Spectral estimates were performed on the first 20 s or 90 s of data in each subject using multi-taper spectral estimate methods (chronux.org). Beta band power was calculated by integrating the spectral power between 10-35 Hz. Correlation between beta band power and DLEP signal power was assessed using Pearson's correlation coefficient, but subject 6 was excluded from this analysis because of prominent 24 Hz noise evident in the LFP spectrum. To examine the time course of beta rhythm suppression by 130 Hz DBS, from 2-55 Hz was band-pass filtered to remove residual stimulation-related artifact and DLEPs and down-sampled to 400 Hz. Multi-taper spectral estimates were then made for each 5 s interval during stimulation and the power in the beta frequency range was plotted across time.

The beta band power suppression across all stimulation conditions was calculated. The final 20 s or 90 s of data for 60 s and 300 s trials, respectively, were selected for analysis across subjects and stimulation conditions to allow for the complete evolution of the effects of DBS during the stimulation epoch. First, the LFP data were high-pass filtered as described above to remove offset. Next, linear interpolation was used in the −0.1-1.5 ms window around the beginning of the amplifier-blanking period to smooth the blanking period and the early DLEP response. Average DLEPs were calculated and subtracted from each individual DLEP. Signals were then band-pass filtered (2-100 Hz) and down-sampled to 400 Hz. These methods effectively removed spectral power at the stimulation frequency and allowed us to quantify the beta band power across stimulation conditions as a percentage of the total power in the 0-90 Hz range. Percent beta band power was normalized by the DBS-OFF percent beta band power across subjects, and a one-way ANOVA was used to test for a significant effect of stimulation frequency on normalized beta band power. Fisher's protected least significant difference test was used for post hoc comparisons between stimulation frequencies.

### Phase-Amplitude Coupling Analysis

Phase-amplitude coupling (PAC) was assessed by calculating the modulation index. Phase amplitude coupling reveals cross-frequency interactions wherein the amplitude of a high frequency signal component is modulated by the phase of a low frequency signal component. Recent experiments have demonstrated significant coupling between beta frequency phase and high frequency oscillation (HFO, >100 Hz) amplitude in patients with Parkinson's disease. In the analysis, ten seconds of LFP data from a DBS-OFF epoch were band-pass filtered (1-500 Hz, 3 pole Butterworth filters), notch filtered at 60 Hz, and down-sampled to 2000 Hz. The conditioned signal was convolved with complex Morlet wavelets (wavelet number 7) with center frequencies ranging from 10-40 Hz (2 Hz interval) and 150-500 Hz (10 Hz interval). Phase, ϕ(t), and amplitude, A(t), were extracted from the low and high frequency convolved data, respectively, and a composite signal z(t)=A(t)e^{iϕ(t)} was constructed for each frequency combination. The modulation index (MI) was quantified from these time series by calculating the mean vector length, MI=|z̅|. A larger mean indicates stronger PAC. Significance was determined by creating a surrogate data set and transforming MI values into z-scores. A set of 200 surrogate composite signals was created by combining A(t) and ϕ(t) with random circular time shifts (>1 s) relative to one another. The mean and standard deviation for the surrogate MI distribution were used to determine the MI z-score for each frequency combination. Significance was defined at α=0.05 corrected for multiple comparisons by dividing by the number of frequency combinations.

The preferred phase for the HFO coupling was visualized by plotting the time-averaged HFO amplitude in 200 ms windows centered at beta rhythm peaks. The conditioned LFP data were bandpass filtered in the beta frequency range (10-30 Hz); then the Hilbert transform was applied and the instantaneous phase of the resulting complex signal was extracted. Beta peaks were identified as instances when the beta phase crossed zero. 200 ms time-series segments centered at the beta peaks were aligned and averaged to get an average beta rhythm time-locked to the beta peak. Instantaneous HFO amplitude was extracted from the LFP data as described above. HFO amplitude data were also windowed and aligned around the beta peaks and averaged across windows. The mean amplitude was finally normalized within each frequency band by subtracting the mean and dividing by the standard deviation of the amplitude distribution across all time.

To calculate the preferred phase of HFO amplitude coupling to the beta rhythm, the conditioned LFP data were bandpass filtered in the beta and HFO ranges and instantaneous phase and amplitude information was extracted from the complex Hilbert transform of the filtered signal. A composite signal was constructed from the HFO amplitude and beta phase time-series as described above. The phase angle of the complex mean of the composite signal indicates the preferred phase of the beta-coupled HFOs. All data analyses were performed using Matlab software.

### Imaging Analysis

Post hoc imaging analysis was performed by two investigators blinded to contact location predictions based on the DLEP and field potential recordings (D.A.T. and P.H.). T1-weighted MR images were obtained with a 3D fast spoiled-gradient-recalled (FSPGR) pulse sequence with an echo time (TE) of 2.5 ms, a repetition time (TR) of 6.5 ms, and flip angle of 12°, at 1 mm isotropic resolution. T2 FLAIR images were acquired with an inversion-prepared gradient echo pulse sequence with a TE of 148 ms, an inversion time (TI) of 2,250 ms, and a TR of 10,000 ms at 1×1 mm in-plane resolution with 1 mm slice thickness and 1 mm spacing between slices.

CT images in the Leksell frame were acquired on a Siemens SOMATOM Definition Flash scanner with a spiral scan using a 512×512 matrix over a 250×250 mm field of view (FOV) for an in-plane resolution of 0.484 mm. Approximately 300 contiguous, non-overlapping slices were acquired covering the entire neurocranium, and slice thicknesses were either 0.6 mm (subjects 1, 2, 6), 1 mm (subject 9), or 5 mm (subjects 3, 4, 5, 7, 8). The tube current and voltage were 250 mA and 120 kVp, respectively. The standard reconstruction process was used.

MR and CT images were merged on a Stealth Framelink workstation, using the CT with the Leksell frame as the “regular” series and the two MR imaging sequences as the alternative series for fusion. The fusion was checked with direct calculation of brain landmarks in Leksell coordinates, to be less than 1.0 mm precision.

### Computational Modeling

Modeling experiments were carried out in the NEURON (7.2) simulation environment with a time step of 0.025 ms. The model contained subthalamic and globus pallidus neurons and afferent cortical axons representing the hyperdirect pathway to STN. Each simulation unit contained ten of each cell type to allow for divergent and convergent projections and intrapallidal inhibition. Model subthalamic and globus pallidus neurons were retrieved from ModelDB (https://senselab.med.yale.edu/modeldb/). These models contain realistic geometry, ion channels, and membrane properties that were derived from experimental studies. Axons from both cell types were modified to be straight and have lengths corresponding to the center-to-center-distance between STN and GPe in humans (˜1.2 cm). Axon diameters were also increased to increase the conduction velocity to match experimental conduction times (˜1-1.2 ms). Cortical axons were identical to the STN and GPe axons. Action potentials at axon terminals triggered synaptic currents in the postsynaptic target. 50 simulation units yielded 500 STN and GPe neurons, and the STN neurons were distributed randomly and uniformly in a 6×2×8 mm prism representing the relevant physical extent of the human STN. Modeling was performed on the Duke Shared Cluster Resource, which allowed parallel computing, thereby saving thousands of hours of time and enabling rapid model development.

### Model Synapses

All synapses were single exponential synapses triggered by presynaptic action potentials with a synaptic delay of 0.5 ms and other parameters selected to match experimental data. Subthalamic neurons receive excitatory, AMPA receptor-mediated synapses from cortex. Ten model AMPA receptor synapses were randomly distributed on subthalamic neuron dendrites, both proximal and distal dendrites with a relative probability of placement of approximately 1:2. Proximal dendrites were defined as dendritic segments within five segments of the soma. AMPA receptor-mediated synapses had a reversal potential of 0 mV and a maximum conductance of 1 pS that decayed with a time constant of 2.5 ms.

The GABAa-mediated inhibitory synapses from GPe to STN are well-characterized, and experimental data was used to guide the selection of synaptic properties. One hundred synapses were distributed on the STN soma (30%), proximal dendrites (40%), and distal dendrites (30%) in accordance with experimental measurements. The reversal potential of the inhibitory synapses was −84 mV and the maximum conductance per synapse was 1 pS. The exponential decay time constant (0.7 ms) was tuned to match the fast component of GPe IPSCs in rat brain slices.

The excitatory and inhibitory synapses within GPe were very similar to those described above. One hundred excitatory synapses from STN to GPe were randomly distributed on distal dendrites. The properties of these synapses were the same as the cortico-subthalamic synapses, except that the decay time constant was 5 ms. Intra-pallidal inhibition on proximal neuronal components is also a key feature in the GPe, and one hundred inhibitory synapses were randomly distributed on pallidal somatic segments.

### Model Stimulation and Recording Contacts

Point electrodes were used to represent the stimulating and recording contacts on the DBS lead. Consistent with the Medtronic 3387 lead used experimentally, the stimulating point source electrode was flanked by the recording electrodes with 3 mm spacing. The ventral recording electrode was placed below the subthalamic nucleus, and the stimulating and dorsal recording electrodes were placed within the STN. This is consistent with the targeted locations of contacts 0-2 during STN lead implant at Duke University Medical Center.

Stimulation was delivered via the point electrode representing contact 1. Symmetric, biphasic current pulses with pulse widths of 100 μs/phase and amplitudes of 3 mA were delivered assuming an infinite, homogenous, isotropic, quasistatic medium with conductivity of 0.3 S/m. Potentials at all the neural elements were calculated using the equation for the potential at a distance (r) away from a point source:

$$φ ( r ) = \frac{I}{4 π \cdot σ \cdot r}$$

where I is the current delivered through the point source, σ is the tissue conductivity, and r is the distance from the current source.

Differential recordings were made from the point electrodes representing contacts 0 and 2 in order to mimic experimental conditions. Neurons that contained a neural element less than 500 μm from any of the point electrode contacts were discarded to mimic the exclusion arising from the DBS lead leaving 311/500 STN neurons contributing to stimulation evoked potentials. Potentials observed at the recording electrodes were calculated as:

$$φ = {\sum }_{k} \frac{{i}_{k}}{4 π \cdot σ \cdot {r}_{k}}$$

where iₖ and rₖ are the transmembrane current and distance from electrode for each STN neural element k; and σ is the tissue conductivity (0.3 S/m). Potentials observed at contact 2 were subtracted from those observed at contact 0 to simulate the desired differential recording (V_{rec}=Φ₀−Φ₂). Recording was simulated with the positive recording contact in STN and the negative recording contact outside of STN by switching the polarity of the recording inputs; this yielded an inverted version of the DLEP.

Any patents or publications mentioned in this specification are indicative of the levels of those skilled in the art to which the disclosure pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the disclosure. Changes therein and other uses will occur to those skilled in the art which are encompassed within the spirit of the disclosure as defined by the scope of the claims.

Referring now to FIGS. 6A through 6I, short latency deep brain stimulation local evoked potentials (DLEPs) evoked by different DBS frequencies at times 5-15 s are shown.

FIG. 6A refers to Subject 1, FIG. 6B to Subject 2, FIG. 6C to Subject 3, FIG. 6D to Subject 4, FIG. 6E to Subject 5, FIG. 6F to Subject 6, FIG. 6G to Subject 7, FIG. 6H refers to Subject 8, and FIG. 6I to Subject 9. Responses for subjects 4, 7, and 8 were digitally inverted.

Referring now to FIGS. 7A through 71, long latency DLEPs evoked by different DBS frequencies at times 5-15 s are shown. FIG. 7A refers to Subject 1, FIG. 7B to Subject 2, FIG. 7C to Subject 3, FIG. 7D to Subject 4, FIG. 7E to Subject 5, FIG. 7F to Subject 6, FIG. 7G to Subject 7, FIG. 7H refers to Subject 8, and FIG. 7I to Subject 9. Subject 9 only received 130 Hz DBS and could not be included. Responses for subjects 4, 7, and 8 were digitally inverted.

Referring now to FIG. 8, computational modeling reveals the neural origin of the DLEPs are shown. (FIG. 8A) The model included subthalamic and globus pallidus neurons and cortical axons of the hyperdirect pathway. (FIG. 8B) The placement of contacts 1 (stimulating) and 2 (negative recording input) was stimulated within the STN and contacts 0 (positive recording input) and 3 ventral and dorsal to STN, respectively. DLEPs calculated from the computational model were similar to those observed clinically for 45 Hz (FIG. 8C) and 130 Hz DBS (FIG. 8D). Post-stimulus time histograms (PSTHs) for STN firing during 45 Hz (FIG. 8E) and 130 Hz (FIG. 8F) DBS reveal a small amount of direct excitation from stimulation and relatively strong inhibition in the interpulse interval with slight increases in firing coincident with the positive phases of the DLEPs. (FIG. 8G, FIG. 8H) GPe spiking is periodic following the strong excitation via STN afferents with peaks in the PSTH at 3-4 ms intervals. The periodic nature of the GPe firing helped shape the DLEP in the model.

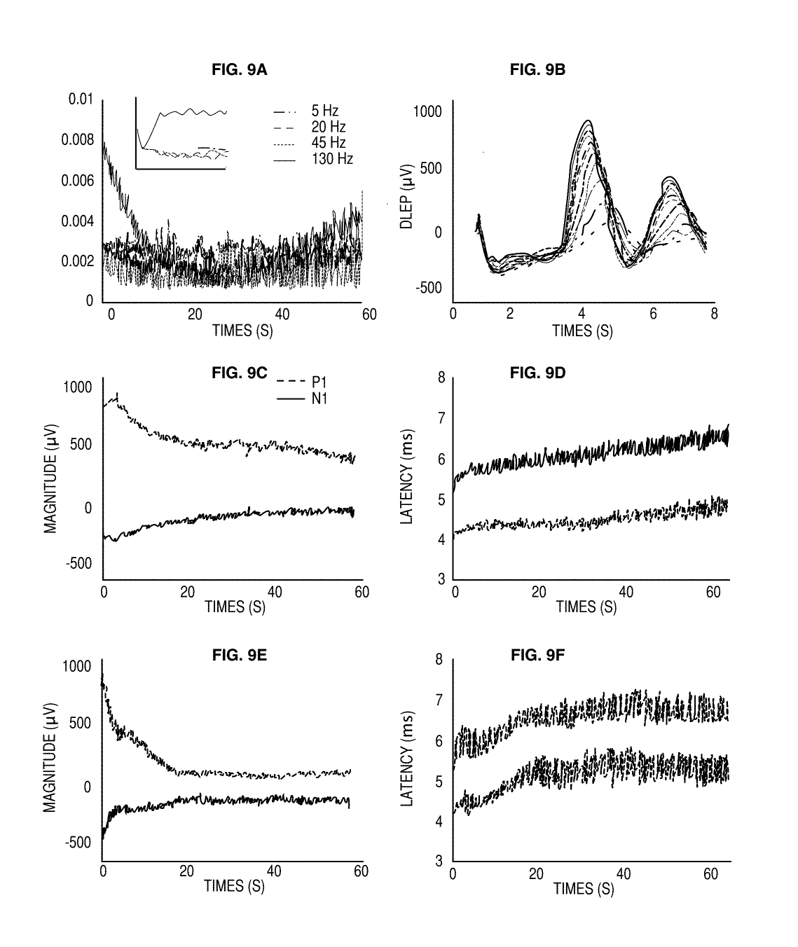

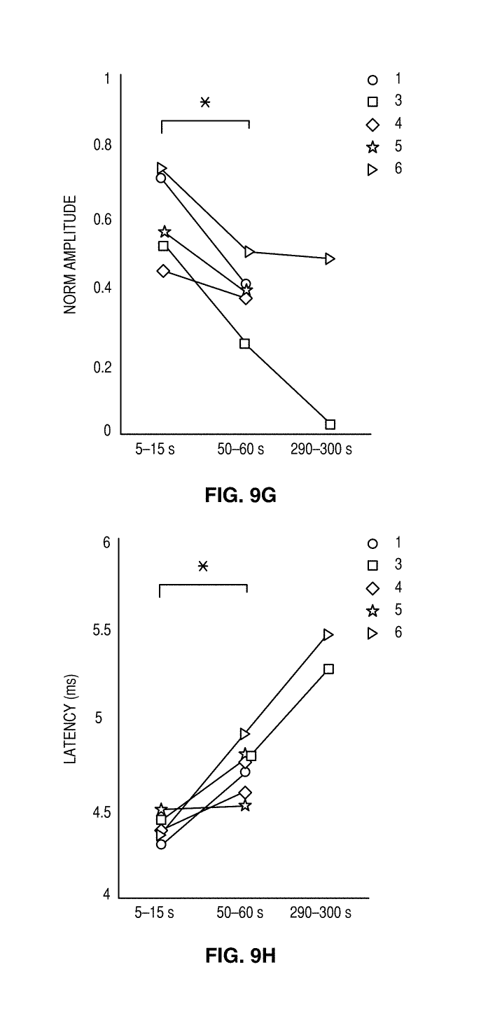

Referring now to FIGS. 9A and 9B, time-dependent changes in DLEPs are shown. (FIG. 9A) The l²-norm of the difference vector between individual DLEPs and the mean DLEP revealed time-dependent changes in the DLEP exclusively for 130 Hz DBS. Rapid changes in the DLEP occurred over the first 100 ms (inset), and slower changes occurred over the remaining stimulation epoch. (FIG. 9B) The amplitude of the P1 peak increased with successive pulses over the first 100 ms. (FIG. 9C, FIG. 9D, FIG. 9E, FIG. 9F) The magnitudes and latencies of the P1 and N1 deflections varied across time. Amplitudes decayed after a brief period of strengthening early in the stimulation epoch; and P1 and N1 latencies increased after initially decreasing in the first 100 ms. Magnitudes and latencies stabilized around 100 s into the stimulation epoch. (FIG. 9H, FIG. 9I) There was a significant decrease in normalized P1 amplitude and a significant increase in P1 latencies between the 5-15 s and 50-60 s stimulation intervals.

Referring now to FIGS. 10A through 10I, Power spectra of local field potentials for each subject are shown. FIG. 10A refers to Subject 1, FIG. 10B to Subject 2, FIG. 10C to Subject 3, FIG. 10D to Subject 4, FIG. 10E to Subject 5, FIG. 10F to Subject 6, FIG. 10G to Subject 7, FIG. 10H refers to Subject 8, and FIG. 10I to Subject 9. Notice the different y-axes across rows. Subject 6 had 24 Hz noise of unknown origin. Subject 8-9 had a noticeable lack of power in the beta range, and these two subjects also lacked DLEPs.

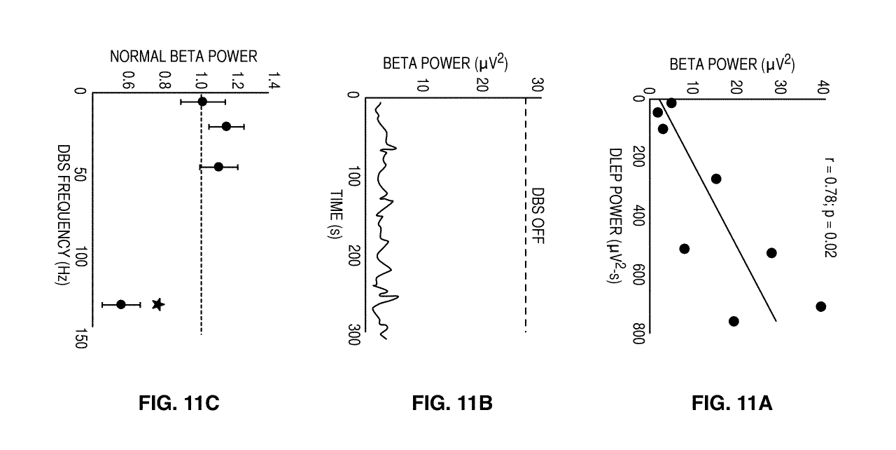

Referring now to FIG. 11, quantification of beta band power are shown. (FIG. 11A) Power in the beta frequency range (10-35 Hz, beta power) was correlated with 130 Hz DLEP power. (FIG. 11B) Beta power was suppressed by 130 Hz DBS and did not vary with time like the DLEP responses (representative data from subject 3 shown). (FIG. 11C) Beta power was normalized within subjects by dividing by the DBS OFF beta power. 130 Hz DBS significantly reduced normalized beta power compared to 5 Hz, 20 Hz, and 45 Hz DBS (p<0.05).

Referring now to FIGS. 12A-12I, phase-amplitude coupling analysis are shown. FIG. 12A refers to Subject 1, FIG. 12B to Subject 2, FIG. 12C to Subject 3, FIG. 12D to Subject 4, FIG. 12E to Subject 5, FIG. 12F to Subject 6, FIG. 12G to Subject 7, FIG. 12H refers to Subject 8, and FIG. 12I to Subject 9. Significant coupling between the phase of beta oscillatory activity and the amplitude of high frequency oscillatory activity was observed in three subjects (1210 1220 1230). The co-modulograms illustrate z-scored modulation indices (MI) across subjects, and significant coupling is highlighted with a white contour. The three subjects with significant phase-amplitude coupling also exhibited more robust beta band peaks in their LFP power spectra.

Referring now to FIG. 13, anti-phase coupling between the beta rhythm and HFO amplitude in subjects with opposite DLEP polarities are shown. Normalized HFO amplitude was time-averaged across windows centered on beta rhythm peaks. (FIG. 13A) Subject 3 showed increased HFO oscillation amplitude near the beta rhythm peak. (FIG. 13B) However, subject 4 showed increased HFO amplitude near beta rhythm troughs. (FIG. 13C) The mean preferred phase of HFOs across subjects confirmed that subject 4 was the only subject with significant PAC that had HFOs that preferentially occurred during the beta rhythm trough. This observation is consistent with the inverted DLEPs observed in subject 4.

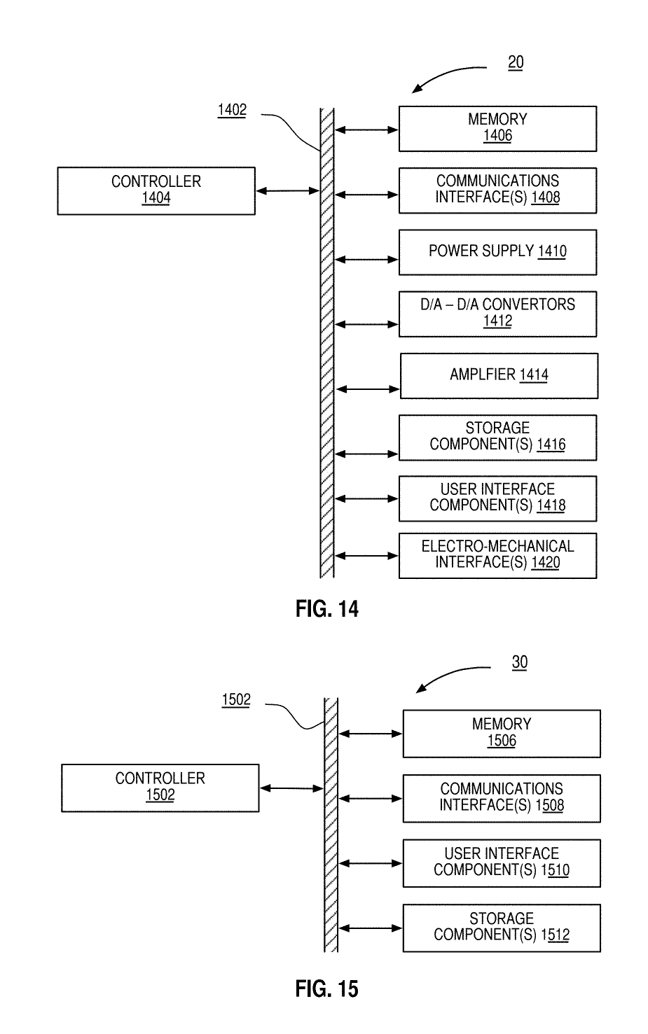

FIG. 14 is a block diagram of a handheld brain stimulation device 20 according to one embodiment of the present disclosure. As illustrated, the handheld brain stimulation device 20 includes a controller 1404 connected to memory 1406, one or more communications interfaces 1408, a power supply 1410, D/A and A/D convertors 1412, an amplifier 1414, one or more storage components 1416, one or more user interface components 1418, and one or more electro-mechanical interface components 1420, by a bus 1402 or similar mechanism. The controller 1404 is a microprocessor, digital ASIC, FPGA, or the like. In general, the handheld brain stimulation device 20 includes a control system having associated memory 1406. In some embodiments, the controller 1404 is a microprocessor, and the brain stimulation modules are implemented in software and stored in the memory 1406 for execution by the controller 1404. However, the present disclosure is not limited thereto. The aforementioned functions and modules may be implemented in software, hardware, or a combination thereof. The handheld brain stimulation device 20 also includes a communication interface 1408 enabling the handheld brain stimulation device 20 to communicate over a network or the like. The power supply is typically a battery. The D/A convertor 1412 operates to convert the electrical signals from digital to analog for subsequent amplification by the amplifier 1414 and attachment to the electrodes. The A/D convertor 1412 operates to digitize locally evoked potential signals. The storage component(s) 1416 is a non-volatile memory operable to store updated/optimized parameters and to store digitized locally evoked potential signals. The one or more user interface components 1418 may comprise a display for presenting signals in real-time and one or more controls operable to receive user settings. The one or more electro-mechanical interface components 1420 operate to provide connection points for electrodes. However, the present invention is not limited thereto.

FIG. 15 is a block diagram of a computing device according to one embodiment of the present disclosure. As illustrated, the computing device 30 includes a controller 1504 connected to memory 1506, one or more communications interfaces 1508, one or more user interface components 1510, and one or more storage components 1512, by a bus 1502 or similar mechanism. The controller 1504 is a microprocessor, digital ASIC, FPGA, or the like. In general, the computing device 30 includes a control system having associated memory 1506. In some embodiments, the controller 1504 is a microprocessor, and the optimization modules are implemented in software and stored in the memory 1506 for execution by the controller 1504. However, the present disclosure is not limited thereto. The aforementioned functions and module may be implemented in software, hardware, or a combination thereof. The computing device 30 also includes a communication interface 1508 enabling the computing device 30 to connect to a network. The one or more user interface components 1510 may include, for example, a touchscreen, a display, one or more user input components (e.g., a keypad), a speaker, or the like, or any combination thereof. The storage component(s) 1512 is a non-volatile memory. However, the present invention is not limited thereto.

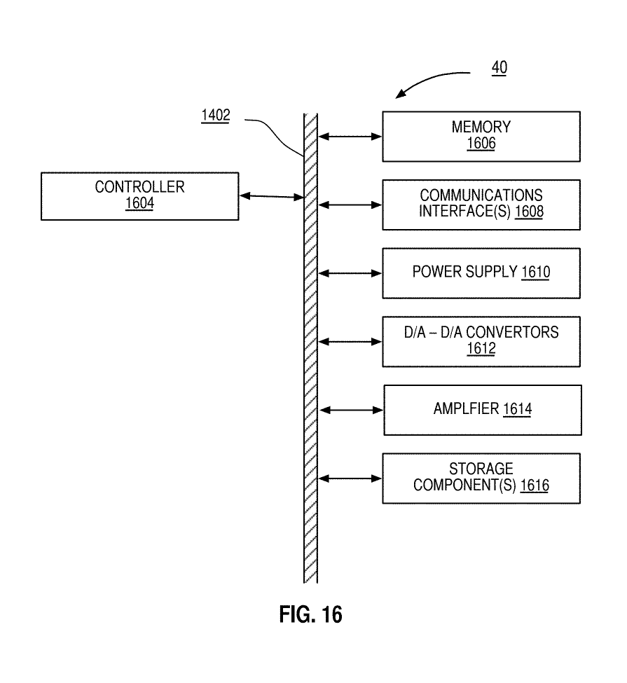

FIG. 16 is a block diagram of an implantable brain stimulation device 40 according to one embodiment of the present disclosure. As illustrated, the implantable brain stimulation device 40 includes a controller 1604 connected to memory 1606, one or more communications interfaces 1608, a power supply 1610, D/A and A/D convertors 1612, an amplifier 1614, and one or more storage components 1616, by a bus 1602 or similar mechanism. The controller 1604 is a microprocessor, digital ASIC, FPGA, or the like. In general, the implantable brain stimulation device 40 includes a control system having associated memory 1606. In some embodiments, the controller 1604 is a microprocessor, and the brain stimulation modules are implemented in software and stored in the memory 1606 for execution by the controller 1604. However, the present disclosure is not limited thereto. The aforementioned functions and modules may be implemented in software, hardware, or a combination thereof. The implantable brain stimulation device 40 also includes a communication interface 1608 enabling the implantable brain stimulation device 40 to communicate over a network or the like. The power supply is typically a battery. The D/A convertor 1612 operates to convert the electrical signals from digital to analog for subsequent amplification by the amplifier 1614 and attachment to the electrodes. The A/D convertor 1612 operates to digitize locally evoked potential signals. The storage component(s) 1616 is a non-volatile memory operable to store updated/optimized parameters and to store digitized locally evoked potential signals. However, the present invention is not limited thereto.

Those skilled in the art will recognize improvements and modifications to the embodiments of the present disclosure. All such improvements and modifications are considered within the scope of the concepts disclosed herein and the claims that follow.

What is claimed is:

1. A device comprising:
a processor and memory, the processor controlling the device to be operable to:
transfer a plurality of stimuli;
receive a plurality of evoked potentials in response to the plurality of stimuli;
perform analysis of the plurality of evoked potentials;
determine one or more stimulation parameters based on the analysis of the plurality of evoked potentials; and
communicate the one or more stimulation parameters.

2. The device of claim 1 in transferring the plurality of stimuli is further operable to:
apply the plurality of stimuli via a corresponding plurality of electrodes.

3. The device of claim 1 in transferring the plurality of stimuli is further operable to:
apply blanking prior to delivery of a pulse; and
apply blanking after the delivery of the pulse.

4. The device of claim 3 in receiving a plurality of evoked potentials is further operable to:
apply clamping using one or more diodes.

5. The device of claim 1 in transferring the one or more stimulation parameters is further operable to:
apply the one or more stimulation parameters via an implantable pulse generator.

6. The device of claim 1, further operable to:
receive selections between stimulation and recording contacts.

7. The device of claim 1, further operable to:
receive adjustments to stimulation parameters and recording parameters.

8. The device of claim 1, wherein the stimulation parameters are one or more items chosen from the group consisting of pulse prior blanking, pulse after blanking, pulse amplitude, pulse width, pulse repetition frequency, and pulse pattern.

9. The device of claim 1 wherein the plurality of evoked potentials are deep brain stimulation locally evoked potential signals.

10. The device of claim 9, further operable to:
analyze the deep brain stimulation locally evoked potential signals.

11. The device of claim 10, further operable to:
process the deep brain stimulation locally evoked potential signals.

12. The device of claim 10, further operable to:
present deep brain stimulation locally evoked potential recordings in real-time.

13. The device of claim 1 in determining one or more parameters based on the analysis of the plurality of evoked potentials is further operable to:
compute one or more predicted locations for one or more contacts.

14. The device of claim 1, further comprising:
a digital to analog convertor coupled to the processor and memory and operable to:
transfer the plurality of stimuli to an amplifier; and
the amplifier coupled to the digital to analog convertor and operable to:
amplify the stimuli; and
an analog to digital convertor coupled to the processor and memory and operable to:
receive the plurality of evoked potentials in response to the plurality of stimuli;
transfer the plurality of evoked potentials to a second amplifier; and
amplify the plurality of evoked potentials.

15. The device of claim 14 further comprising:
a battery coupled to the processor and memory and operable to:
power the device.

16. The device of claim 15, wherein the device is an implantable device.

17. The device of claim 15 further comprising:
a display coupled to the processor and the memory and operable to:
present a signal; and
an electromechanical interface coupled to the processor and the memory and operable to:
couple electrodes to the device.

18. The device of claim 17 further comprising:
a first control coupled to the processor and memory and operable to:
receive adjustments to pulse width; and
a second control coupled to the processor and memory and operable to:
receive adjustments to pulse amplitude; and
a third control coupled to the processor and memory and operable to:
receive adjustments to pulse repetition frequency; and
a fourth control coupled to the processor and memory and operable to:
receive adjustments to pulse repetition pattern; and
a fifth control coupled to the processor and memory and operable to:
receive a selection between recording presentation mode and stimulation display presentation mode.

19. A method comprising:
transferring a plurality of stimuli;
receiving a plurality of evoked potentials in response to the plurality of stimuli;
performing analysis of the plurality of evoked potentials;
determining one or more stimulation parameters based on the analysis of the plurality of evoked potentials; and
communicating the one or more stimulation parameters.

20. A system comprising:
a device comprising:
a processor and memory operable to:
transfer a plurality of stimuli;
receive a plurality of evoked potentials in response to the plurality of stimuli;
perform analysis of the plurality of evoked potentials;
determine one or more stimulation parameters based on the analysis of the plurality of evoked potentials; and
communicate the one or more stimulation parameters to a client; and
the client operable to:
receive the one or more stimulation parameters from the device; and
present the the one or more stimulation parameters on a display of a second device executing the client.
